(19) United States
(12) Patent Application Publication
(10) Pub. No.: US 2023/0053346 A1
(43) Pub. Date: Feb. 23, 2023

(54) DEVICE PERFORMANCE PREDICTION USING MATERIAL PROPERTIES

(71) Applicant: KROMEK GROUP, PLC, Sedgefield (GB)

(72) Inventors: Ian Radley, Sedgefield (GB); Handong Li, Pittsburgh, PA (US); Brian William Harris, Butler, PA (US); Carl D. Brunetta, Greensburg, PA (US)

(21) Appl. No.: 17/893,682

(22) Filed: Aug. 23, 2022

Related U.S. Application Data
(60) Provisional application No. 63/235,985, filed on Aug. 23, 2021.

(51) Int. Cl. G01N 23/18; G01N 23/083; G01N 23/046

(52) U.S. Cl. CPC G01N 23/18; G01N 23/083; G01N 23/046; G01N 2223/50; G01N 2223/04; G01N 2223/419

(57) ABSTRACT

One embodiment provides a method for predicting the performance of a device based upon parameters of an underlying material, comprising: measuring a predetermined parameter of a material to be used in manufacturing the device; identifying, from a value generated from the measuring, a value of a property of the material; and determining a predicted performance of the device by correlating the value of the property to a performance value. Other aspects are described and claimed.

### CROSS REFERENCE TO RELATED APPLICATION

This application claims priority to U.S. Provisional Patent Application Ser. No. 63/235,985, filed on Aug. 23, 2021, and entitled “DEVICE PERFORMANCE PREDICTION USING MATERIAL PROPERTIES,” the contents of which are incorporated by reference herein.

### FIELD

This application relates generally to device production, and, more particularly, to predicting the performance of a device based upon the properties of the materials used within the device.

### BACKGROUND

Many electronic devices, for example, photon detectors, and other imaging devices, have particular performance requirements which designate how the device should operate. Utilizing performance requirements ensures that the devices will all perform within a desired performance range which can be used as a basis for determining how well an overall device may perform. Some imaging devices or imaging applications, for example, Spectral Computed Tomography (CT) applications, have very demanding requirements on the performance of devices used with the applications, for example, the semiconductor detectors' performance under high-flux x-ray radiation. These requirements include good energy spectral and temporal response, good long-term and short-term stability, good input/output count rate linearity, and low noise. Thus, it is critical to ensure that the imaging devices will have the desired performance requirements.

### BRIEF SUMMARY

In summary, one aspect provides a method for predicting the performance of a device based upon parameters of an underlying material, including: measuring a predetermined parameter of a material to be used in manufacturing the device; identifying, from a value generated from the measuring, a value of a property of the material; and determining a predicted performance of the device by correlating the value of the property to a performance value.

Another aspect provides an apparatus for predicting the performance of a device based upon parameters of an underlying material, including: a memory device that stores instructions executable by the processor to: measure a predetermined parameter of a material to be used in manufacturing the device; identify, from a value generated from the measuring, a value of a property of the material; and determine a predicted performance of the device by correlating the value of the property to a performance value.

A further aspect provides a product for predicting the performance of a device based upon parameters of an underlying material, including: a storage device that stores code, the code being executable by a processor and comprising: code that measures a predetermined parameter of a material to be used in manufacturing the device; code that identifies, from a value generated from the measuring, a value of a property of the material; and code that determines a predicted performance of the device by correlating the value of the property to a performance value.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a transient curve of TOF measurement.

FIG. 2 illustrates a CT detector is short-term stability vs. de-trapping ratio (tailing ratio).

FIG. 3 illustrates a CT detector pile-up loss vs. de-trapping ratio (tailing ratio).

FIG. 4 illustrates a CT detector peak to valley ratio vs. de-trapping ratio (tailing ratio).

FIG. 5 illustrates a CT detector peak location vs. de-trapping ratio (tailing ratio).

FIG. 6 illustrates a CT detector 10s short-term stability vs. de-trapping ratio (tailing ratio).

FIG. 7 illustrates an example method of predicting a device performance based upon measurement of a material parameter.

FIG. 8 illustrates an example of information handling device circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

The example of a photon detector will be used here throughout. However, it should be understood that the described system and method can be applied to any device that it made from materials such as semiconductor materials, lab-grown materials, natural materials, and the like. For many imaging devices, for example, photon detectors which are made from materials such as semiconductor material it is difficult to know how the device will perform before the imaging device is complete. In other words, until the photon detector has been completely manufactured, it is difficult to know how well the detector will perform or the performance characteristics of the detector. Thus, after the detector has been manufactured, rigorous device characterizations and testing of the device have to be performed. If the detector does not meet the performance requirements during the characterization and testing, then the processing resources required to manufacture the devices from the base materials is wasted. Stated differently, any loss of yield or device performance results in wasted time and resources, including both the raw material resources and any resources used to make the final device. Thus, a method to predict the device performance of a device before the device is made is desirable. Specifically, it is desirable to be able to predict the performance of a device based upon characteristics of the raw or base material used within the device before the device is manufactured. Additionally, characterization of devices and materials requires resources, so it is also desirable to minimize the level of characterization that is required.

Accordingly, the described system and method provides a technique of predicting device performance utilizing the measurement of specific material parameters which are correlated to final device performance. In one particular embodiment, the method predicts performance of photon detectors by the measurement of properties of the detection material. A more specific embodiment predicts the performance of semiconductor-based photon detectors by the measurement of specific parameters of the semiconductor material. The system measures a predetermined parameter of an underlying material (i.e., a material used in the manufacturing of a device). From the value generated from the measurement of the predetermined parameter, the system identifies a value of a property of the material. From the value of the property of the material the system can determine a predictor performance of the device by correlating the value of the property to device performance values.

Thus, the described system and method provides a technical improvement over current device performance identification techniques. Rather than having to completely manufacturer the device and then identifying the performance characteristics, the described system and method is able to predict device performance based upon parameters and properties of the underlying materials used in manufacturing the device. Thus, instead of wasting time and resources on devices that may end up being unusable due to the performance characteristics of the device, the described system and method increases the chances that the device will have the desired performance characteristics once manufactured, thereby reducing waste.

The illustrated example embodiments will be best understood by reference to the figures. The following description is intended only by way of example, and simply illustrates certain example embodiments.

One technique for predicting the performance of a semiconductor-based photon detector utilizes the carrier de-trapping analysis. This analysis may also utilize the time-of-flight (TOF) measurement technique. TOF measurements have been used in semiconductor material and device characterizations to study the carrier transports and electric field profile with minimal perturbation of the intrinsic transport properties of the material. In other words, the TOF measurement technique allows for the determination of material properties without changing or otherwise affecting the properties of the material. This method utilizes alpha particle or light radiation to generate the charges that move across the device thickness under the bias.

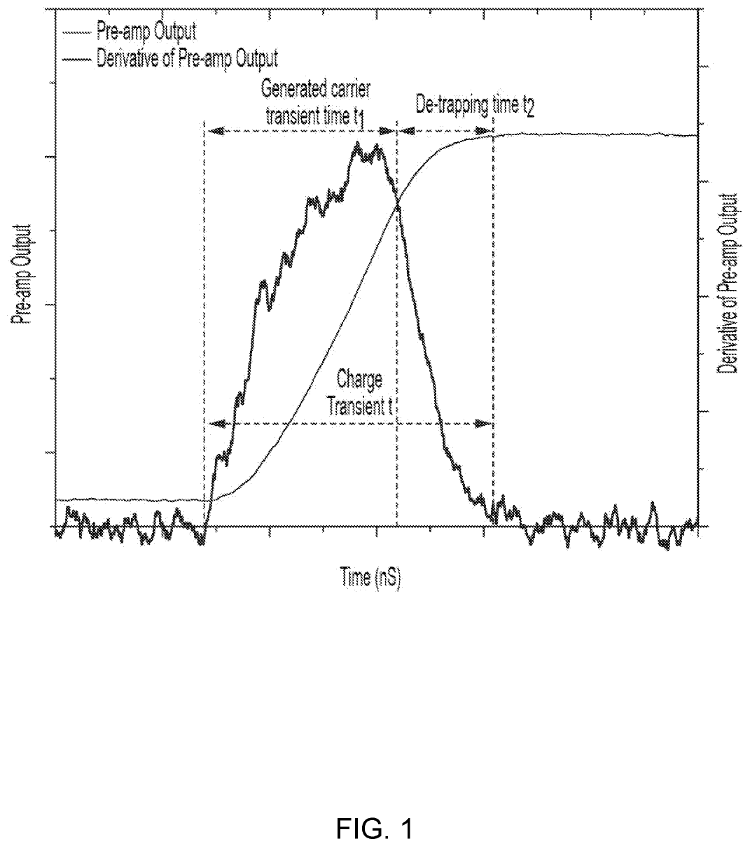

A typical signal from a TOF measurement from a cadmium-zinc-telluride (CdZnTe or CZT) semiconductor high energy photon detector is shown in FIG. 1. CZT is one type of semiconductor material that can used in the manufacture of photon detectors. Other types of semiconductor materials may also be used, for example, cadmium-telluride (CdTe), germanium, thallium bromide, or the like. Each of these materials have their own unique measurement graphs. In the measurement in FIG. 1, the transient time t of the charge needs to be determined among other information. Since the charge arriving time cannot be easily identified for some types of materials from original preamp output signals, the derivation of the signal is applied (also shown in FIG. 1). With known bias and detector thickness, the effective mobility of the electron and the hole are obtained this way with proper bias polarity. In other words, some parameters of the material are known or can be easily determined in order to identify properties of the material that are indicative of device performance.

The effective mobility measured this way is highly bias dependent for some semiconductor materials, such as defect dependent CZT materials. More detailed analysis on the TOF signal from these materials shows that the induced signal includes the part from most of the generated carrier, represented by a transient time t₁, and the part from the de-trapped carrier with an additional transient de-trapping time t₂. For some semiconductor materials, the de-trapping time t₂ is a sensitive indicator of material defect density and could consist of a significant part of the whole transient time. This is the main reason leading to the effective mobility's bias dependence.

In identifying a value of the property and using the transient times example, the system may perform a calculation using the transient times. Since a TOF measurement technique is being utilized and since the thickness of the material is known, the system may use a formula for calculating the defect density. In other words, the TOF measurement provides a value for how long it takes the charge to travel through the material. Since the thickness of the material is known, the defect density of the material is one, if not the only, remaining material properties that will affect the transient times. Thus, the longer the charge takes to travel through the material, the greater the defect density within the material.

In a specific embodiment, the de-trapping ratio (defined as t₁/(t₁+t₂) in percent), is a material property indicator on the material's defect density and correlated to detector performance, especially in high-flux x-ray detectors. In other words, in this embodiment, the system utilizes a TOF measurement technique to measure a parameter of the material, specifically, a transient time and a transient de-trapping time. From these measured transient times, the system identifies a value of a property of the material, specifically, a defect density of the material. The defect density can then be correlated to a performance value, thereby providing a predicted performance for the device utilizing the material.

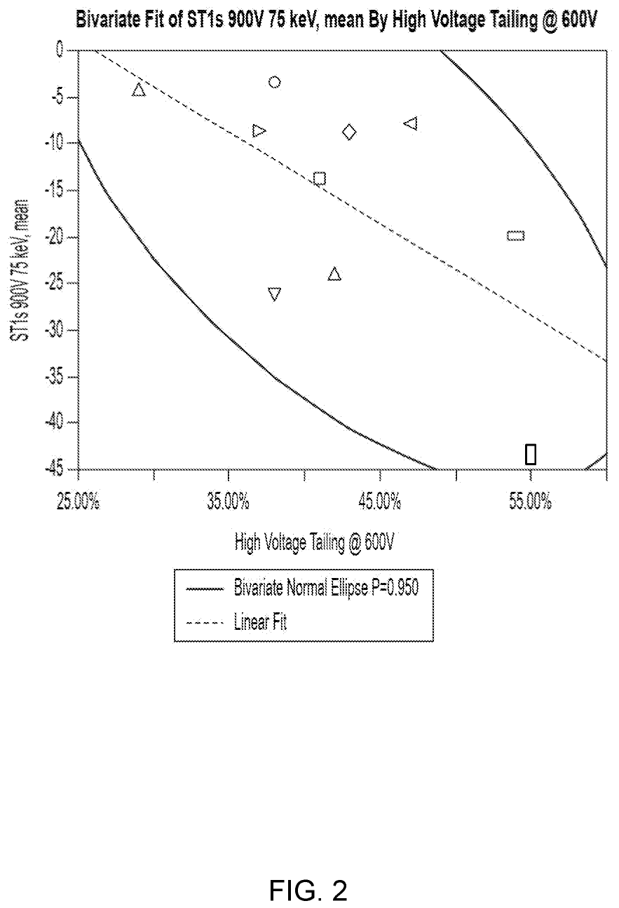

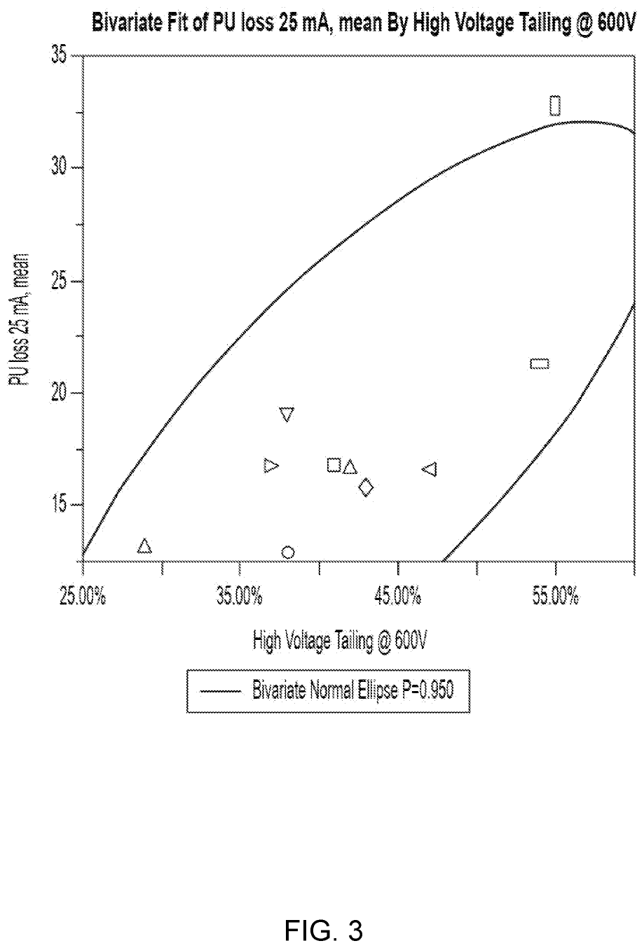

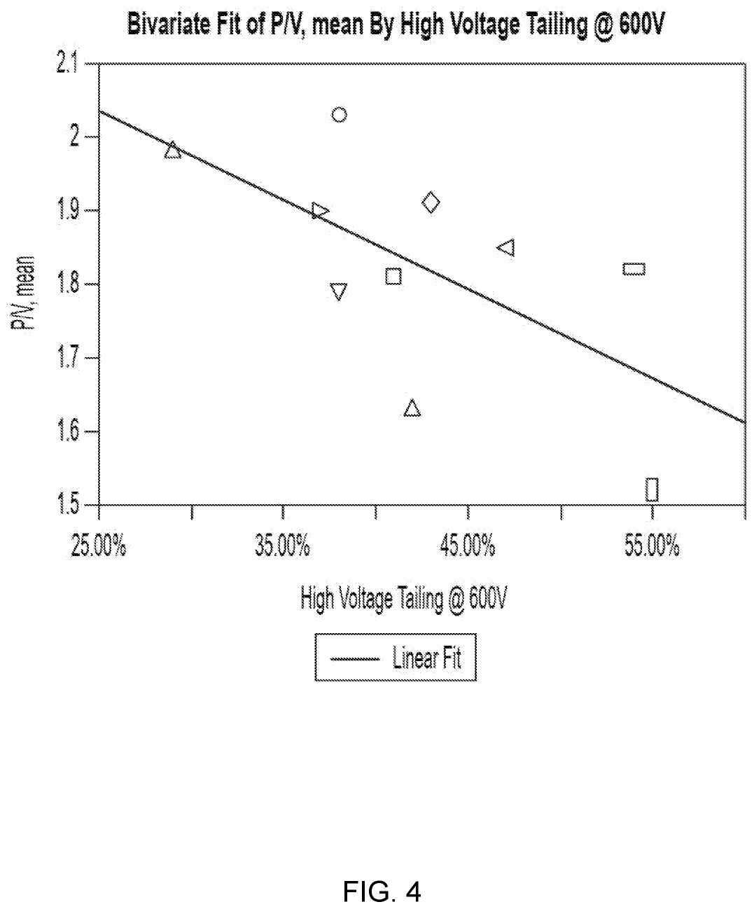

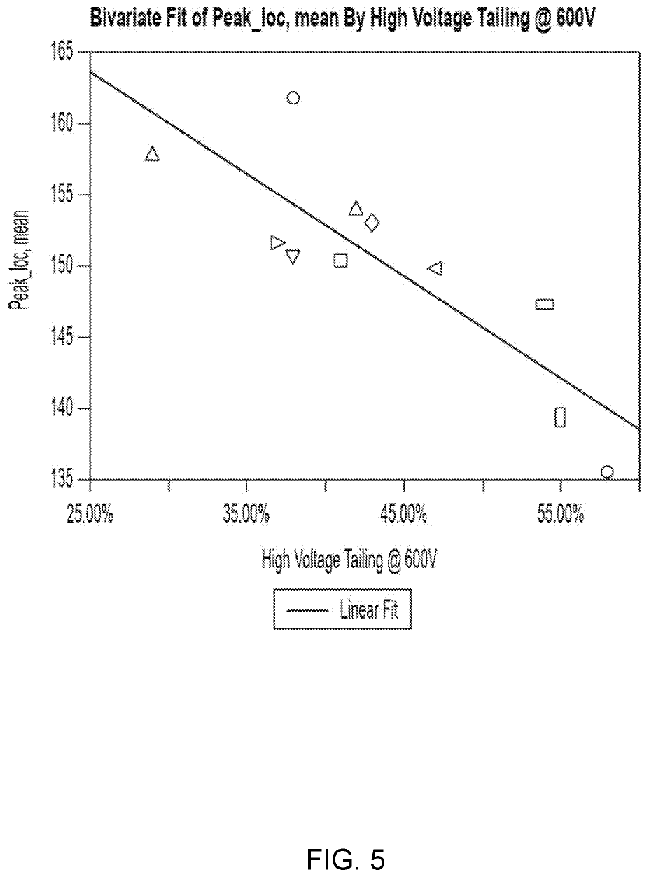

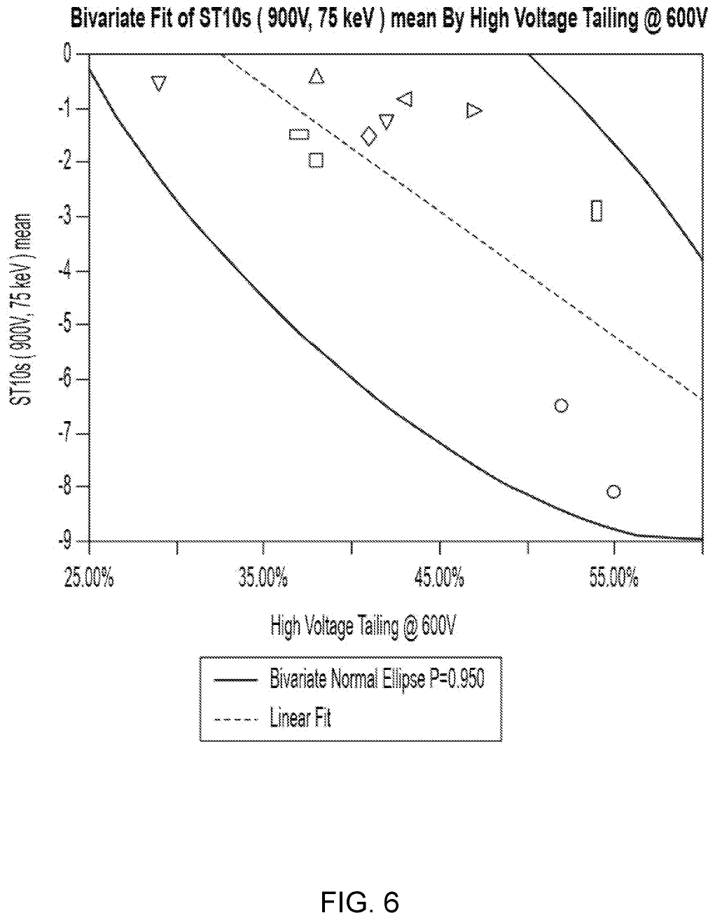

Due to its simple sample configuration, easy test setup and data processing, and non-destructive nature, the described technique can be efficient and cost-effective for materials and device characterizations. FIG. 2-FIG. 6 illustrate some additional data from some of the CT detectors' performance parameters and their de-trapping ratio. From the data, the material de-trapping ratios give good predictions on the detector's performance: higher tailing ratio materials always show lower spectral CT performance. FIG. 2 illustrates a CT detector is short-term stability vs. de-trapping ratio (tailing ratio). FIG. 3 illustrates a CT detector pile-up loss vs. de-trapping ratio (tailing ratio). FIG. 4 illustrates a CT detector peak to valley ratio vs. de-trapping ratio (tailing ratio). FIG. 5 illustrates a CT detector peak location vs. de-trapping ratio (tailing ratio). FIG. 6 illustrates a CT detector 10s short-term stability vs. de-trapping ratio (tailing ratio).

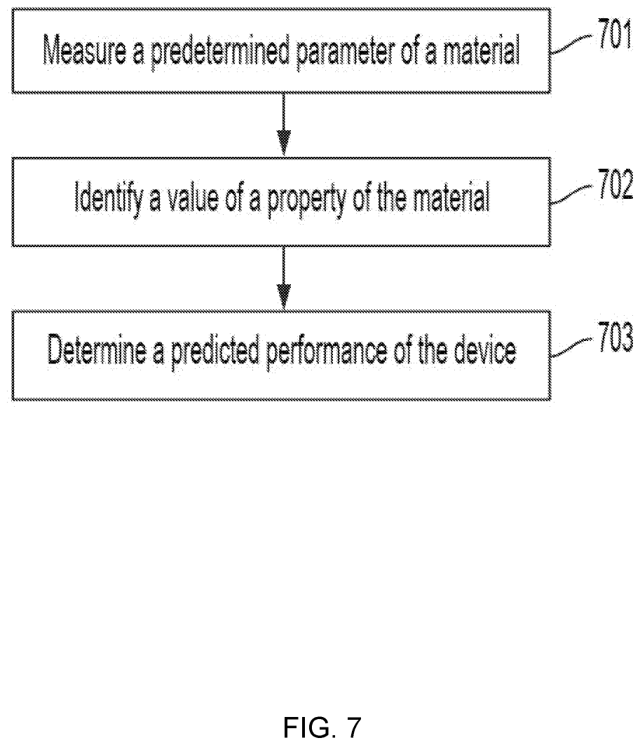

FIG. 7 illustrates an example method for predicting the performance of a device based upon parameters of an underlying material. At 701, the system measures a predetermined parameter of a material to be used in manufacturing the device. At 702, the system identifies, from a value generated from the measuring, a value of a property of the material. At 703, the system determines a predicted performance of the device by correlating the value of the property to a performance value.

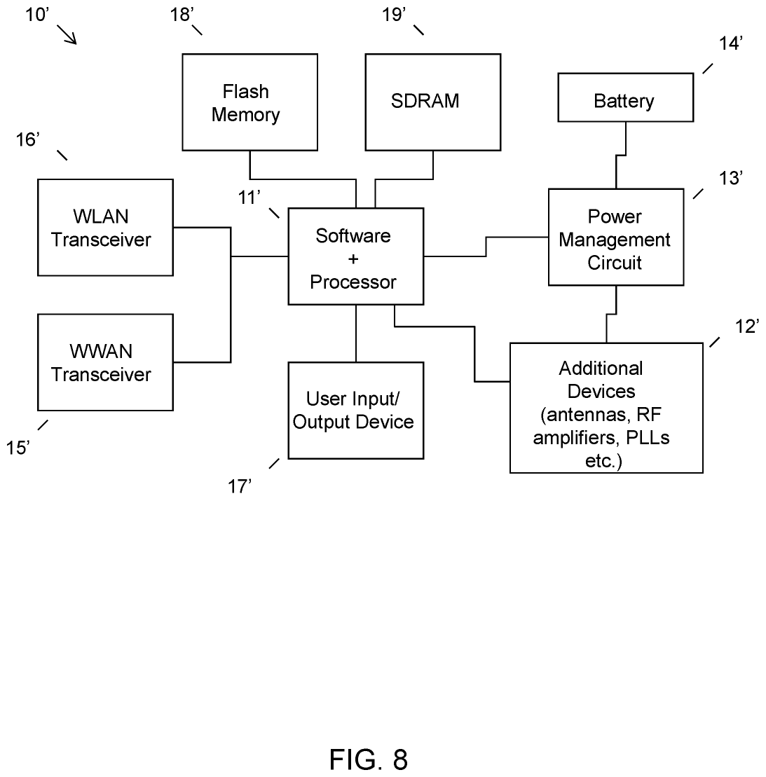

While various other circuits, circuitry or components may be utilized in information handling devices, with regard to an instrument for predicting device performance utilizing the measurement of specific material parameters which are correlated to final device performance according to any one of the various embodiments described herein, an example is illustrated in FIG. 8. Device circuitry 10′ may include a measurement system on a chip design found in, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment of an instrument for determining a characteristic of an electron cloud upon a subset of pixels.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and “non-transitory” includes all media except signal media.

Program code embodied on a storage medium may be transmitted using any appropriate medium, including but not limited to wireless, wireline, optical fiber cable, RF, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for predicting the performance of a device based upon parameters of an underlying material, comprising:
measuring a predetermined parameter of a material to be used in manufacturing the device;
identifying, from a value generated from the measuring, a value of a property of the material; and
determining a predicted performance of the device by correlating the value of the property to a performance value.

2. The method of claim 1, wherein the measuring comprises measuring an electric field of the material utilizing a time of flight measurement technique.

3. The method of claim 1, wherein the measuring comprises utilizing a carrier de-trapping analysis.

4. The method of claim 1, wherein the property comprises a defect density of the material.

5. The method of claim 1, wherein the device comprises a photon detector.

6. The method of claim 1, wherein the identifying is determined using known values of parameters other than the predetermined parameter.

7. The method of claim 1, wherein the material comprises a semiconductor material.

8. The method of claim 1, wherein the material comprises a cadmium zinc telluride.

9. The method of claim 1, wherein the measuring comprises measuring an effective mobility of an electron and a hole with a proper bias polarity based upon a known bias and a known detector thickness.

10. The method of claim 2, wherein the time of flight measurement technique comprises a transient time and a transient de-trapping time.

11. An apparatus for predicting the performance of a device based upon parameters of an underlying material, comprising:
a memory device that stores instructions executable by the processor to:
measure a predetermined parameter of a material to be used in manufacturing the device;
identify, from a value generated from the measuring, a value of a property of the material; and
determine a predicted performance of the device by correlating the value of the property to a performance value.

12. The apparatus of claim 11, wherein the measuring comprises measuring an electric field of the material utilizing a time of flight measurement technique.

13. The apparatus of claim 11, wherein the measuring comprises utilizing a carrier de-trapping analysis.

14. The apparatus of claim 11, wherein the property comprises a defect density of the material.

15. The apparatus of claim 11, wherein the device comprises a photon detector.

16. The apparatus of claim 11, wherein the identifying is determined using known values of parameters other than the predetermined parameter.

17. The apparatus of claim 11, wherein the material comprises a semiconductor material.

18. The apparatus of claim 11, wherein the material comprises a cadmium zinc telluride.

19. The apparatus of claim 11, wherein the measuring comprises measuring an effective mobility of an electron and a hole with a proper bias polarity based upon a known bias and a known detector thickness.

20. A product for predicting the performance of a device based upon parameters of an underlying material, comprising:
a storage device that stores code, the code being executable by a processor and comprising:
code that measures a predetermined parameter of a material to be used in manufacturing the device;
code that identifies, from a value generated from the measuring, a value of a property of the material; and
code that determines a predicted performance of the device by correlating the value of the property to a performance value.
